(19) United States
(12) Patent Application Publication
(10) Pub. No.: US 2025/0125018 A1
(43) Pub. Date: Apr. 17, 2025

(54) SYSTEMS, METHODS, AND DEVICES FOR MICROSTRUCTURE CHARACTERIZATION

(71) Applicant: Board of Regents, The University of Texas System, Austin, TX (US)

(72) Inventors: Binata Joddar, El Paso, TX (US); Wilson Poon, El Paso, TX (US); Ivana Hernandez, El Paso, TX (US); Salma P. Ramirez, El Paso, TX (US)

(73) Assignee: Board of Regents, The University of Texas System, Austin, TX (US)

(21) Appl. No.: 18/910,334

(22) Filed: Oct. 9, 2024

Related U.S. Application Data
(60) Provisional application No. 63/589,519, filed on Oct. 11, 2023.

(51) Int. Cl. G16C 20/50; G01N 33/543; G01N 33/553; G06T 17/00; G16C 20/80

(52) U.S. Cl. CPC G16C 20/50; G01N 33/54346; G01N 33/553; G06T 17/00; G16C 20/80; G06T 2200/24

(57) ABSTRACT

Systems, methods, and devices disclosed herein include three-dimensional bioprinting technologies which produce cell scaffolds with a high degree of complexity and precision. A bioprinting ink mixture can be formed with coated gold nanoparticles in an alginate-gelatin solution. Upon bioprinting a 3D scaffold microstructure from the bioink mixture, an imaging procedure is performed on the 3D scaffold microstructure using the coated gold nanoparticles as a contrasting agent. A microstructure characterization is determined from the imaging procedure, and a virtual 3D reconstruction of the microstructure characterization can be generated for presentation on a graphical user interface (GUI) of a computing device. These techniques can be used to determine whether the resultant internal core of the microstructure properly mimics the architecture of the native tissue extracellular matrix (ECM) present in-vivo by determining the degree of pore interconnectivity which can improve cell distribution, attachment, and growth in the 3D scaffold microstructure.

### CROSS REFERENCE TO RELATED APPLICATION

This application claims priority to U.S. Provisional Patent Application Ser. No. 63/589,519, filed Oct. 11, 2023, and titled “SYSTEMS, METHODS, AND DEVICES FOR MICROSTRUCTURE CHARACTERIZATION,” which is incorporated by reference herein in its entirety.

### ACKNOWLEDGEMENT OF GOVERNMENT SUPPORT

This invention was made with government support under grant number HL154511 awarded by The National Institutes of Health. The government has certain rights in the invention.

### BACKGROUND

### 1. Technical Field

Aspects of the presently disclosed technology generally relate to tomography imaging techniques and, more specifically, to nanoparticle contrasting agents for micro computed tomography.

### 2. Discussion of Related Art

Tissue engineering attempts to create in vitro three-dimensional (3D) tissues resembling native tissues for regenerative medicine in vivo. While 3D bioprinting has potential for mimicking human tissue complexity, characterizing the bioprinted microstructure is challenging due to the small scale and dense, irregular structures of the bioprinted microstructure. It is with these observations in mind that the presently disclosed technology was conceived.

### SUMMARY

Systems, methods, and devices disclosed herein can address the aforementioned issues. For example, a method to characterize a microstructure can include providing a three-dimensional (3D) scaffold microstructure, the 3D scaffold microstructure comprising coated gold nanoparticles; performing an imaging procedure on the 3D scaffold microstructure using the coated gold nanoparticles as a contrasting agent; determining a microstructure characterization based on the imaging procedure; and/or generating a reconstruction of the microstructure characterization. The reconstruction of the microstructure characterization may be a virtual 3D reconstruction of the microstructure characterization that is presented at or generated by a graphical user interface (GUI) of a computing device. The reconstruction of the microstructure characterization may be a 3D reconstruction of the microstructure characterization that is presented at or printed by a 3D printer. In some examples, providing the 3D scaffold microstructure includes bioprinting the 3D scaffold microstructure using a 3D bioprinter. The 3D scaffold microstructure can include an alginate-gelatin hydrogel. Furthermore, the coated gold nanoparticles can include methoxy-poly (ethylene glycol) (methoxy-PEG) coated gold nanoparticles. Also, the method can comprise determining, based on the microstructure characterization, a degree of pore interconnectivity affecting cell distribution, attachment, or growth in the 3D scaffold microstructure. Additionally, the imaging procedure can include a micro-computed tomography (micro-CT) procedure. Moreover, the method can include determining a size of gold nanoparticles by performing at least one of an ultraviolet (UV)-visible spectroscopy procedure, a dynamic light scattering (DLS) procedure, a zeta potential measurement procedure, a scanning electron microscopy procedure (SEM), or a transmission electron microscopy (TEM) procedure. Providing the 3D scaffold microstructure can also include obtaining a solution of 2 kDa methoxy-poly (ethylene glycol) (methoxy-PEG) coated gold nanoparticles, mixing the solution of the 2 kDa methoxy-PEG coated gold nanoparticles with a saline solution, a first concentration of gelatin, and a second concentration of sodium alginate to form a resultant solution, and/or providing the resultant solution to a 3D bioprinter operating under one or more predefined 3D bioprinting parameters. The first concentration of gelatin can be between 4-6% (w/v) gelatin and the second concentration of sodium alginate can be between 6-8% (w/v) sodium alginate. Additionally, the one or more predefined 3D bioprinting parameters can include at least one of a nozzle size of 22G, a printing speed of about 1 mm/s, a pressure of about 25 kPA, or a temperature of about 25° C. The method can also include optimizing a concentration of the coated gold nanoparticles in the 3D scaffold microstructure to optimize cell growth while optimizing imaging contrast.

In some examples, a method to characterize a microstructure includes providing a three-dimensional (3D) scaffold microstructure, the 3D scaffold microstructure includes methoxy-poly(ethylene glycol) (methoxy-PEG) coated gold nanoparticles; performing an imaging procedure on the 3D scaffold microstructure using the methoxy-PEG coated gold nanoparticles as a contrasting agent; and/or determining a microstructure characterization based on the imaging procedure, a virtual 3D reconstruction for presentation at a graphical user interface (GUI) of a computing device is based on the microstructure characterization.

In some examples, the methoxy-PEG coated gold nanoparticles can have a core diameter of between 15 nm and 200 nm. Additionally, the methoxy-PEG coated gold nanoparticles can have a core diameter of about 60 nm. In some examples, the providing of the 3D scaffold microstructure can include preparing a solution of 2 kDa methoxy-PEG coated gold nanoparticles, diluting the solution to form varying concentrations of the solution, and/or using the varying concentrations of the solution to form, via a 3D bioprinter, a plurality alginate-gelatin hydrogels having varying concentrations of the 2 kDa methoxy-PEG coated gold nanoparticles.

In some examples, a method to characterize a microstructure can include providing a three-dimensional (3D) scaffold microstructure, the 3D scaffold microstructure includes methoxy-poly(ethylene glycol) (methoxy-PEG) coated gold nanoparticles; performing an imaging procedure on the 3D scaffold microstructure, using the methoxy-PEG coated gold nanoparticles as a contrasting agent, to determine a microstructure characterization of the 3D scaffold microstructure; and/or causing a virtual 3D reconstruction to be presented at a graphical user interface (GUI) of a computing device based on the microstructure characterization.

In some examples, providing the 3D scaffold microstructure can include bioprinting, using a 3D bioprinter, the 3D scaffold microstructure from a solution of the methoxy-PEG coated gold nanoparticles, gelatin, and sodium alginate. A gold nanoparticle characterization procedure can include at least one of an ultraviolet (UV)-visible spectroscopy procedure, a dynamic light scattering (DLS) procedure, a zeta potential measurement procedure, or a transmission electron microscopy (TEM) procedure. Additionally, the method can further include determining, based on the microstructure characterization, a degree of pore interconnectivity affecting cell distribution, attachment, or growth in the 3D scaffold microstructure. In some scenarios, the method can further include optimizing a concentration of the methoxy-PEG coated gold nanoparticles in the 3D scaffold microstructure to optimize cell growth while optimizing imaging contrast to generate or yield an optimized concentration of the methoxy-PEG coated gold nanoparticles in a solution that is between a 0.022 optical density and a 2.2 optical density.

Other implementations are also described and recited herein. Further, while multiple implementations are disclosed, still other implementations of the presently disclosed technology will become apparent to those skilled in the art from the following detailed description, which shows and describes illustrative implementations of the presently disclosed technology. As will be realized, the presently disclosed technology is capable of modifications in various aspects, all without departing from the spirit and scope of the presently disclosed technology. Accordingly, the drawings and detailed description are to be regarded as illustrative in nature and not limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates an example microstructure characterization system using coated gold nanoparticles as an imaging contrasting agent.

FIG. 2 illustrates an example microstructure characterization system including characterization results for coated gold nanoparticles.

FIG. 3 illustrates an example microstructure characterization system including characterization results of gold nanoparticles in cell culture conditions.

FIG. 4 illustrates an example microstructure characterization system including characterization results of a 3D bioprinted scaffold microstructure.

FIG. 5 illustrates an example microstructure characterization system including characterization results of coated gold nanoparticles in cell culture conditions.

FIG. 6 illustrates an example microstructure characterization system including a 3D bioprinted scaffold microstructure.

FIG. 7 illustrates an example microstructure characterization system including a virtual 3D reconstruction of a 3D bioprinted scaffold microstructure.

FIG. 8 illustrates an example microstructure characterization system including mechanical properties characterizations of 3D bioprinted scaffold microstructures.

FIG. 9 illustrates an example microstructure characterization system including a network environment and one or more computing devices.

FIG. 10 illustrates an example method for microstructure characterization which can be performed by any of the systems depicted in FIGS. 1-9.

FIG. 11 illustrates an example of a Scanning Electron Microscopy (SEM) image of 3D bioprinted scaffolds microstructures with 200 nm core-sized gold nanoparticles.

FIG. 12A-12D are fluorescent microscopic images showing live-dead assay results for: samples containing 200 nm core-sized AuNPs (FIG. 12A); samples containing 200 nm core-sized AuNPs at 1:1,000 dilution (FIG. 12B); samples containing 200 nm core-sized AuNPs at 1:10,000 dilution (FIG. 12C); and samples containing 200 nm core-sized AuNPs at 1:5,000 dilution (FIG. 12D).

It will be apparent to one skilled in the art after review of the entirety disclosed that the steps illustrated in the figures listed above may be performed in other than the recited order, and that one or more steps illustrated in these figures may be optional.

### DETAILED DESCRIPTION

Certain aspects of the presently disclosed technology are directed to systems, methods, and devices of tissue engineering to promote tissue repair or regeneration. The techniques can use a combination of scaffolds with viable cells and biomolecules to create 3D tissues in-vitro that can recapitulate all aspects of living tissues in-vivo. As such, the system(s) disclosed herein can be used for design and development of scaffolds that can promote and enhance these functions. The biomanufactured tissue scaffolds can possess a microstructure similar to the natural extracellular matrix of healthy tissues. Although it may be difficult to characterize and visualize 3DBP scaffold microstructure, particularly hydrogel-based scaffolds, the techniques disclosed herein address this problem.

In some examples, the systems disclosed herein include three-dimensional bioprinting (3DBP) technologies with cell scaffolds produced with a high degree of complexity and precision. A resultant internal/core microstructure can mimic the architecture of the native tissue extracellular matrix (ECM) present in-vivo. The scaffolds can possess high pore interconnectivity to allow cells to distribute, attach, and grow in the structure.

In some examples, the systems can provide a characterization technique for presenting and/or studying microstructure of the 3DBP scaffolds which can be scaled up for many biomanufacturing systems. Advanced high-resolution imaging techniques such as micro-computed tomography (micro-CT) can provide imaging capabilities with isotropic resolutions that range from 100 nm to a few millimeters, which may be used for imaging a thick cell scaffold. Because cell-based hydrogel scaffolds are comprised of more than 70% aqueous environments, their imaging can be challenging. The micro-CT technique disclosed herein can be used to image scaffolds embedded with contrast agents such as gold nanoparticles (Au-NPs), which can provide higher contrast and resolution. These techniques can be helpful for studying the microstructure of the cell-based scaffold in order to predict its mechanical fidelity and biodegradability for tissue engineering applications. The systems can determine the optimized concentration of Au-NPs to be integrated into the 3DBP scaffolds to sustain cell growth and promote tissue regeneration while providing sufficient contrast for high resolution microscopic imaging. The system can use micro-CT scanning to explore the microstructure of 3DBP alginate-gelatin scaffolds incorporated with varying concentrations of Au-NPs; and characterize and model the scaffold's mechanical properties. Herein, optimum concentrations of Au-NPs for encapsulation within 3DBP hydrogel-based scaffolds can be determined which provides both adequate contrast enhancement in micro-CT scanning and sustained biocompatibility of the encapsulated cells. The systems can compare the scaffold's microstructure and mechanical properties via experimental and modeling techniques.

In some examples, by applying micro-CT imaging of cell-based scaffolds, the presently disclosed technology can quickly adapt imaging techniques to the fast-moving pace of biomanufacturing of engineered tissues and create new knowledge and research products in this field. Results outputted by these systems can optimize the workflow of 3DBP of cardiac tissues using a combination of tools from other disciplines that include structural mechanics-based microstructure modelling and advanced micro-CT based image analysis. Outputs can be sent to downstream systems and APIs for data integration and usage by other systems (e.g., display systems, wireless networks, updates to tracker systems, a report generator, and so forth). This technology can include tools and approaches for enhancing bio-additive manufacturing science.

In some examples, this application of micro-computed tomography (micro-CT) as a characterization technique for the study and investigation of the microstructure of 3D scaffold structures can be produced via 3DBP. The system can include preparation, characterization, and cytotoxicity analysis of Au-NPs incorporated into 3DBP hydrogels for micro-CT evaluation. The Au-NPs can be characterized using various techniques, including UV-Vis spectroscopy, dynamic light scattering (DLS), zeta potential measurement, and transmission electron microscopy (TEM). The characterization results can confirm an amount or degree of coating and/or whether the coating is successful of the Au-NPs with 2kDa methoxy-PEG, and can reveal their shape (e.g., a spherical shape) and/or other dimensions, such as a mean core width or diameter (e.g., of 66 nm). The systems disclosed herein can perform cytotoxicity analysis using live-dead fluorescent microscopy which can indicate that all tested Au-NP solutions are non-toxic to AC16 cells in both 2D and 3D culture conditions. Scanning electron microscopy (SEM) can show distinguishable differences in image contrast and intensity between samples with and without Au-NPs, with high concentrations of Au-NPs displaying nanoparticle aggregates. Micro-CT imaging can demonstrate that scaffolds containing Au-NPs depict enhanced imaging resolution and quality, providing improved visualization of the microstructure. The systems can also generate a 3D reconstruction of scaffold structures from micro-CT imaging using rendering software (e.g., DragonFly Software), which results in improved visualization presentations. Additionally or alternatively, updates can be sent to tracker systems monitoring a status or health of the cells in the microstructure.

In some examples, a mechanical analysis can reveal that the addition of coated Au-NPs enhanced the mechanical properties of acellular scaffolds. Additionally, the presence of cells can lead to biodegradation and/or reduced mechanical strength over time. By successfully preparing and characterizing Au-NPs with their non-toxic nature in both 2D and 3D culture conditions, the disclosed technology can positively influence imaging quality and the impact on the mechanical properties of 3D-printed hydrogels. These results can contribute to the development of functional and biocompatible materials for tissue engineering and regenerative medicine applications.

In some examples, the biomanufactured tissue scaffolds possess a microstructure similar to the natural extracellular matrix of healthy tissues, yet 3DBP can yield non-homogenous pore distribution and this, in turn, can affect cell viability and tissue function. Although it may be difficult to characterize and visualize 3DBP scaffold microstructure, particularly hydrogel-based scaffolds, the systems disclosed herein can use micro-CT to assess the microstructure of 3D bioprinted scaffolds with coated Au-NPs. Mechanical analysis can also reveal enhanced mechanical properties in acellular scaffolds. This technology can be applied to better understand how to develop new 3DBP materials and biomanufacturing approaches, thereby contributing to regenerative medicine advancements. The resulting materials and products can be monitored, tracked, used for further research, and/or can be implanted into a living subject. As such, the improved imaging techniques disclosed herein can be a valuable tool for optimizing tissue-engineered scaffold, leading to more successful tissue regeneration and repair.

Additional benefits and advantages of the disclosed technology will become apparent from the detailed description below.

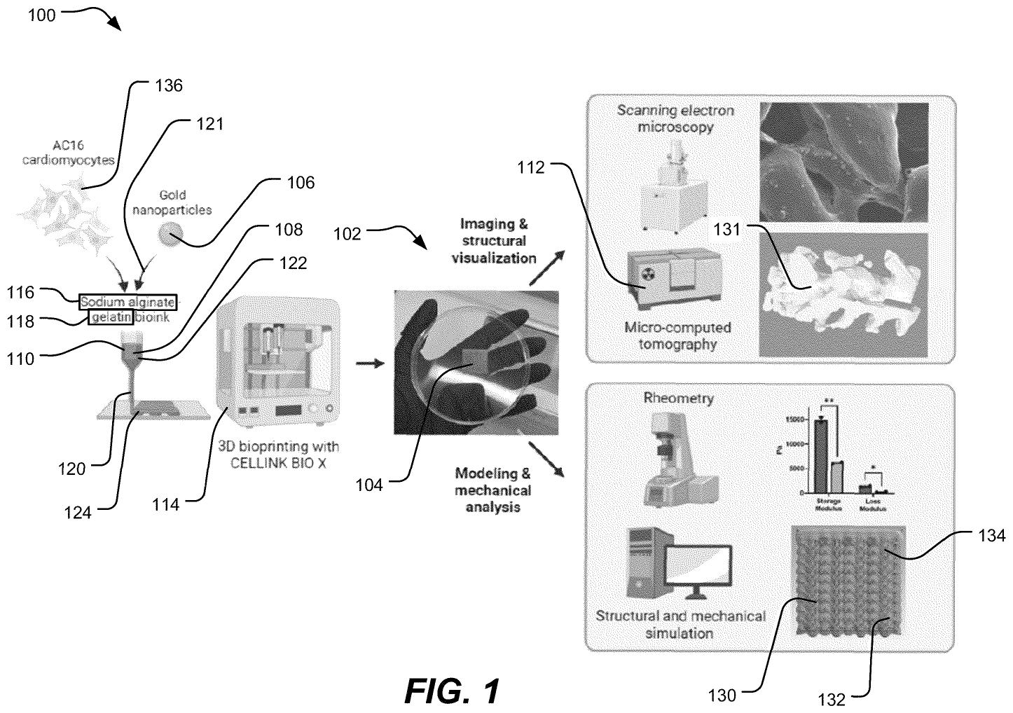

FIG. 1 depicts an example microstructure characterization system 100 including a 3D microstructure 102 (e.g., which can be a 3D scaffold microstructure 104) using coated gold nanoparticles (Au-NPs) 106 incorporated into a hydrogel 108 or other base material 110. The Microstructure characterization system 100 can also include a micro-computed tomography (micro-CT) system 112 for studying and investigating the 3D scaffold microstructure 104 produced using a 3D Bioprinting (3DBP) machine 114. As noted above, the incorporation of the coated Au-NPs 106 into the 3DBP hydrogel 108 can enhance imaging resolution, quality, and provide for visualization of the microstructure.

In some examples, to make the 3DBP hydrogel 108, medium viscosity sodium alginate 116 and gelatin 118 (e.g., from porcine skin) can be obtained (e.g., from MP Biomedicals of Solon, OH, US), which can form the primary or the main components of a bioink mixture 120 for bioprinting. The coated Au-NPs 106 can be 2 kDa methoxy-PEG coated Au-NPs with core diameter of 60 nm plus or minus 10 nm, or in a range of 15 nm to 200 nm, and can be added to a concentration of solution for bioprinting (e.g., a 2200 optical density (OD) concentration obtainable from Luna Nanotech of Markham, ON, Canada). Moreover, other lengths of PEG and/or other terminal groups of PEG may be used for coating the Au-NPs.

In some instances, to prepare the bioink mixture 120 for the 3DBP machine 114, a solution of 2 kDa methoxy-PEG coated Au-NPs 121 can be diluted 1:1000, 1:10,000 and 1:100,000 in molecular grade water to prepare solutions of varying concentrations (e.g., 2.2 OD, 0.22 OD, and 0.022 OD respectively). These Au-NP solutions 122 can be characterized with UV-visible spectroscopy, dynamic light scattering, zeta potential measurement, and transmission electron microscopy. Cell cytotoxicity studies can also be performed on the Au-NP solutions 122 with AC16 human cardiomyocyte cell lines.

In some scenarios, Au-NP alginate-gelatin based hydrogel 124 can be made by mixing 1 μL of the Au-NP solutions 122 with dilutions of 1:10,000 and 1:100,000 to 1000 μL of phosphate buffered saline for a homogenous distribution. Thereafter, gelatin 118 being in a range of w/v percentages, such as 1%-10%, (e.g., 4%, 5%, or 6%) and sodium alginate 116 in a range of w/v percentages, such as 1%-10%, (e.g., 6%, 7%, or 8%) can be dissolved in the Au-NP solutions 122. For cellular scaffolds 134, a sub portion such as half of the total volume of the resultant solution can be composed of AC16 cardiomyocyte cell growth medium along with phosphate buffered saline (PBS).

In some examples, the 3D scaffold microstructure 104 can be designed using Fusion360 Software. 3DBP acellular structures 130 can be printed using an accordion-like design 132 (20 mm×20 mm×1 mm), for example, on a 100 nm diameter petri dish as the printing base. After the 3D structures are printed, they can be cross-linked with 100 mM calcium chloride.

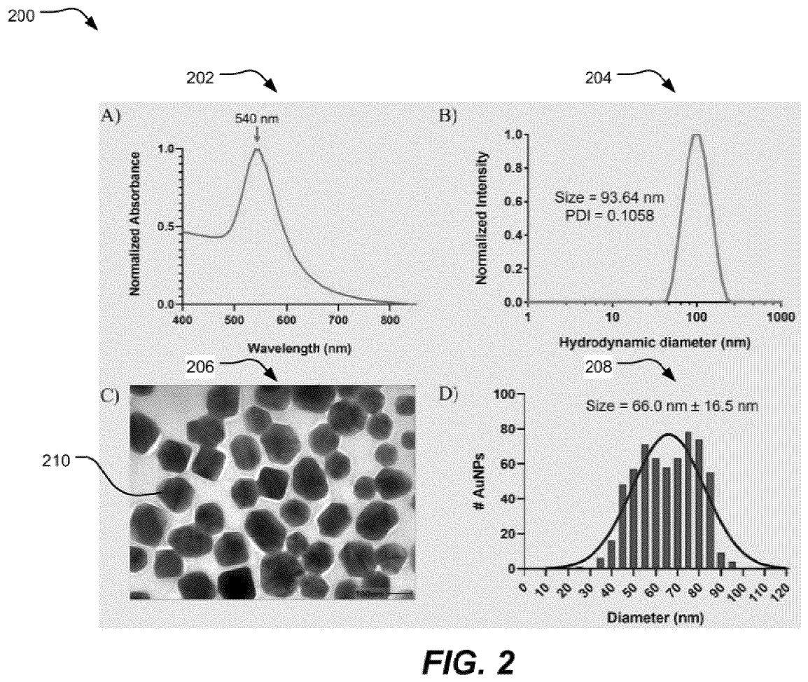

FIG. 2 depicts an example microstructure characterization system 200 including (A) a Normalized absorbance spectra of Au-NPs 202 as determined by UV-Vis spectroscopy; (B) a Hydrodynamic diameter of Au-NPs 204 as determined by dynamic light scattering (DLS); (C) a Representative TEM micrograph 206 of 2 kDa methoxy-PEG coated Au-NPs, with a scale bar of 100 nm; and (D) Histogram and Gaussian fits of the measured Au-NP size distribution as determined from TEM micrographs 208 (n=6000 nanoparticles).

In some examples, the microstructure characterization system 200 can use various materials and methods to construct the hydrogels 108 and the coated Au-NPs 106. For making the 3DBP hydrogels 108, medium viscosity sodium alginate 116 and gelatin 118 can be used as the main components of the bioink mixture 120 for bioprinting.

In some scenarios, the UV-visible spectroscopy can be performed from 400-850 nm using a UV-Vis Spectrophotometer (e.g., the NanoDrop One™ Microvolume UV-Vis Spectrophotometer from Thermo Fisher Scientific of Waltham, MA, United States). A maximum peak absorbance can be used to calculate the concentration of the samples using Beer-Lambert Law. Additionally, a molar absorption coefficient of the 60 nm Au-NPs can be obtained from a product datasheet provided by the vendor. The hydrodynamic size, polydispersity index (PDI), and zeta potential of the Au-NPs 106 can be measured, for example, using the Zetasizer Ultra Red (Malvern Panalytical, Malvern, UK). 1 mL of the Au-NP solutions 122 in micro cuvettes can be measured using the backscatter detection mode in order to obtain the hydrodynamic size and the PDI of the Au-NP solutions. Nanoparticle size and distribution can be reported by intensity. 1 mL of the gold nanoparticle solution 122 can be added into a folded capillary zeta cell to measure the zeta potential of Au-NPs. In some scenarios, the gold nanoparticles can be in a range of 15nm to 200 nm in diameter. Smaller gold nanoparticles may have higher dispersion in the 3D bioink mixture 120 and may create a more uniform image (e.g., due to less or no aggregation). That said, in some instances, smaller gold nanoparticles may have less contrast so an overall amount of gold nanoparticles in the bioink mixture 120 can be increased (e.g., which may have a different toxicity profile for particular cell lines). Larger gold nanoparticles may have less dispersion and/or a higher contrast, so a lower amount of gold nanoparticles can be used. By using these optimization techniques, a concentration and/or size of the coated gold nanoparticles in the 3D scaffold microstructure can be optimized for both cell growth and imaging contrast.

In some examples, a characterization of the Au-NP solutions 122 can include performing a TEM 208 to visualize the overall morphology and size distribution of the NPs. The Au-NPs 210 can be air dried on a plasma-treated TEM copper grid. Negative staining can be done with 0.2% uranyl acetate. Au-NPs 210 can be imaged, for example, using a JEOL 2010F 200 kV field emission analytical TEM with a Direct Electron DE-12 camera.

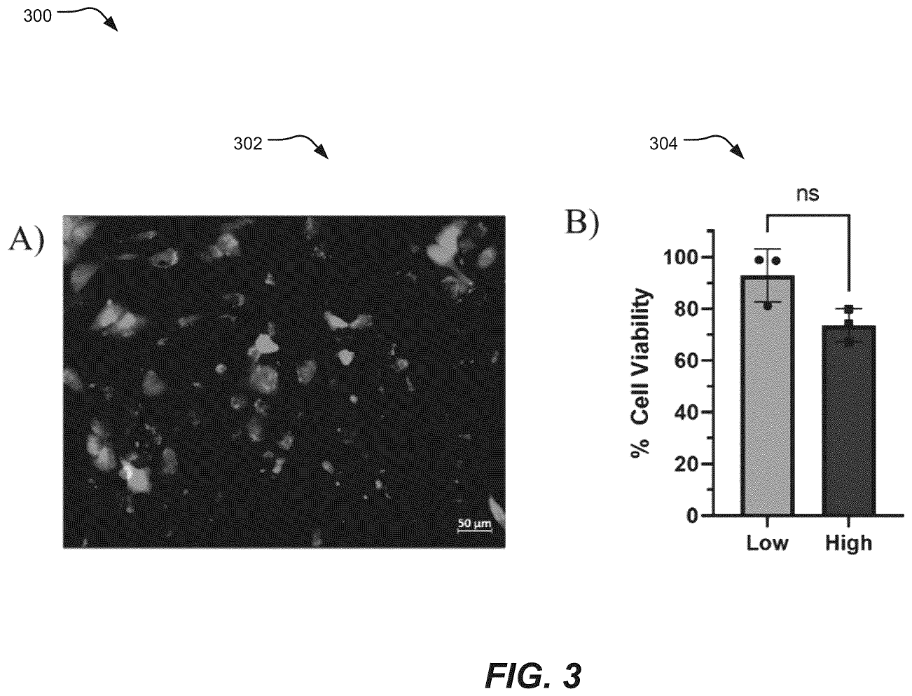

In some examples, the microstructure characterization system 200 can perform an assessment of the in vitro biocompatibility of the Au-NPs 210. AC16 human cardiomyocyte cell lines (e.g., SCC109, EMD Millipore, MA) can be cultured in Dulbecco's Modified Eagle's Medium/Nutrient Mixture (e.g., DMEM/F12, Sigma Cat. No. D6434, St. Louis, MO, United States) 10% FBS (EMD Millipore Cat. No. ES-009-B), and/or 1× penicillin-streptomycin solution (EMD Millipore Cat. No. TMS-AB2-C). 1 μL of the Au-NP solution 122 can be added into a 6-well culture plate containing AC16 cells, with a density of approximately 60,000 cells per well and cultured for 2 days in an incubator under standard cell culture conditions. Cytotoxicity of the Au-NPs 210 can be measured using a Live-Dead Assay Kit (e.g., Thermo Fisher Scientific, USA) used according to the protocol provided by the vendor. Cell viability can be quantified using the formula below. Calcein AM (e.g., shown as green in FIG. 3) stained live cells, while ethidium homodimer (e.g., shown as red in FIG. 3) can be used to stain dead cells after being incubated in the samples for at least 1 hour at room temperature (RT) (25° C.). By way of example, a % Cell viability (% live cells) versus % of dead cells can be quantified using the following formula:

$$No . of live / dead cells \% = \frac{Number of live or dead cells}{Total numbers of live + dead cells} \times 10$$

In some scenarios, the microstructure characterization system 200 can perform a preparation procedure to prepare the Alginate-Gelatin-Au-NP hydrogels. For example, to make the Au-NP alginate-gelatin based hydrogels 124, 1 μL of the gold nanoparticle solution 122 in the dilutions of 1:10,000 (high) and 1:100,000 (low) can be added to 1000 μL of Phosphate Buffered Saline (1×) (Cytiva, Marlborough, MA, United States), and the Au-NPs 210 can be mixed for a homogeneous distribution. Thereafter, 4%-6% or 5% (w/v) gelatin 118 and 6%-8% or 7% (w/v) sodium alginate 116 can be dissolved in the solution 122 premixed with Au-NPs 210. Furthermore, for cellular scaffolds 134, a portion such as half of the total volume of the resultant solution can be composed of AC16 cardiomyocyte cell growth medium along with PBS.

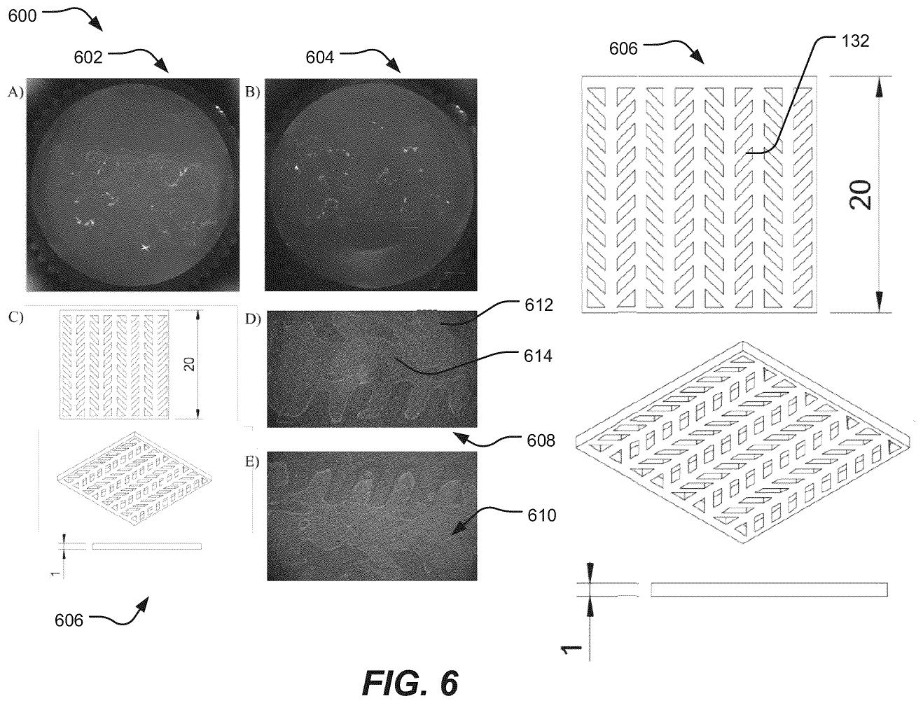

In some examples, the microstructure characterization system 200 can perform a Three-Dimensional Bioprinting (3DBP) procedure. For example, to make the 3DBP hydrogels 124, STL. files can be designed using 3DBP software (e.g., Fusion360). The 3DBP acellular structures 130 can be printed using the accordion-like design 132 (20 mm×20 mm×1 mm) and can include a plurality of protrusions 612 (e.g., parallel protrusions) extending from a base extension 614 (e.g., FIG. 6). The petri dish (e.g., 100 mm diameter) can be used as the printing base. Using a CELLINK BIO X 3D bioprinter (Blacksburg, VA), the 3DBP acellular structures 130 can be made using the following parameters depicted in Table 1:

TABLE 1

| Nozzle Size | Printing Speed | Pressure | Temperature |
|---|---|---|---|
| 22 G | 1 mm/s | 45 kPa | 25° C. |

After the 3DBP acellular structures 130 are printed, they can be cross-linked with a range of 80 mM to 120 mM calcium chloride (e.g., 80, mM, 100 mM, or 120 mM) by pipetting 1000 μL of the solution into the petri dish. The crosslinking solution can be left in the petri dish for 15 minutes, after which the solution can be removed, and the 3DBP acellular structures 130 can be rinsed thrice with 1×PBS.

In some examples, the microstructure characterization system 200 can perform a Scanning Electron Microscopy (SEM) procedure. For example, an SEM can be used as a preliminary screening tool to study the 3DBP acellular structures 130 which can assist in choosing the optimal concentrations of Au-NP solutions 122 for micro-computed tomography. Samples with Au-NPs 210 can generate enhanced contrast, resolution, and quality in a dose-dependent manner in comparison with samples without Au-NPs 210. Furthermore, if the SEM image from a particular sample set depicts enhanced contrast and resolution, the microstructure characterization system 200 can determine that the particular dose of Au-NPs 210 reveals the similar enhancement in image quality when analyzed using micro-CT.

In some scenarios, to do this, two different Au-NP concentrations (1:10,000 (high) and 1:100,000 (low)) can be used to print the 3DBP acellular structures 130. SEM images can be obtained, for example, using a Hitachi SU3500 Variable-Pressure Scanning Electron Microscope (Santa Clara, CA, United States). Optimization of the concentration of the Au-NPs in the 3DBP scaffolds for micro-CT can be based on SEM results obtained. In some scenarios, obtained SEM micrographs can be analyzed using their intensity profile in ImageJ using the following sequence under “analyze”>“histogram”>“intensity”. From the obtained values, the mean intensity can be calculated and reported as a Mean+Standard deviation (AU) data value.

In some instance, the microstructure characterization system 200 can perform a Micro-CT for 3D microstructure visualization procedure. For example, 3DBP acellular structures 130 containing two different Au-NP concentrations (e.g., high and low) can be bioprinted immediately before micro-CT imaging. In addition, 3DBP cellular scaffolds 134 containing AC16 cardiomyocytes 136 with either low or high concentrations of Au-NPs 210 can also be bioprinted immediately before micro-CT imaging. To prepare the samples for micro-CT imaging, samples with the dimensions of 2 mm×2 mm×1 mm can be isolated from all scaffolds, can be held with 1×PBS three times, and/or fixed with 4% paraformaldehyde (PFA) (15 min, RT) and can be scanned in distilled water. The parameters which can be used for the micro-CT scans are depicted in Table 2.

TABLE 2

| X-ray Settings | Value |
|---|---|
| Voltage (kV) | 70 |
| Power (W) | 8.5 |
| Filter | No filter |
| Acquisition Time (s) | 0.05 |
| Source-Object Distance (mm) | 20.378 |
| Detector-Object Distance (mm) | 170.125 |

In some examples, 16-bit TIFF images can be reconstructed (e.g., by Xradia Reconstructor) with a voxel size of 8.00 μm. Some or all images can be obtained as slices 706 from each sample and each image slice can be processed, for example, using ImageJ software. The processed images can be used to create a z-stacked projection obtained by combining multiple image slices along the z-axis of the entire scaffold structure for both the acellular and cellular samples. The z-stacked projections can be used to extract maximum intensity images for all samples studied. These images can represent the combination image of the slices with the highest intensity values, improving the 3D scaffold structure visualization 131. ImageJ software can be further used to delineate the visible 3D scaffold structure as a region of interest (ROI) from the images obtained for both the acellular and cellular samples. This step can be used to enhance the visibility and quality of the 3D scaffold structure visualization 131. The 3D volumetric reconstruction of the scaffold structure (e.g., the DBP acellular structures 130 and/or the cellular scaffolds 134) based on the micro-CT data and images can be performed using DragonFly Software (Montreal, Quebec) which can use the obtained image data to reconstruct a 3D representation of the scaffolds. This software can be used for segmenting an acellular and a cellular scaffold from micro-CT images. The processes involved for visualizing the structure of the scaffolds in 3D and extracting it can include using a 2D region of interest (ROI) painter tools. The microstructure characterization system 200 can use a feature of the software called ‘multi-slice’, which can accelerate the process of ROI segmentation. Once the scaffolds are segmented, a 3D representation of the scaffold can be generated, which can be converted into a volumetric mesh to be used for further mechanical evaluation. This can provide an in-depth analysis of the scaffold to gain valuable insights into the physical and mechanical properties of the 3DBP structure.

In some examples, the microstructure characterization system 200 can perform a rheological analysis procedure. For example, both 3DBP acellular structures 130 and cellular 3DBP scaffolds can be analyzed to evaluate their mechanical properties. For the cellular scaffolds 134, AC16 cells can be mixed in the bioink mixture 120 to constitute a final cell seeding density of 9×105 cells/mL and printed to study the rheological properties of cell-based scaffolds in comparison with acellular controls. Rheometric analysis of the samples can be conducted on an Anton-Paar MCR 92 rheometer (Anton-Paar, Austria) with a PP25/S measuring system and a 25 mm parallel plate with a 1 mm gap between the plate and the stage. The scaffold material's linear viscoelastic range (LVE) can be within a strain range of 0.1 to 150% at a constant frequency of 1 Hz. From this evaluation, an optimal strain within the linear viscoelastic region can be selected by the microstructure characterization system 200 to be kept constant during a frequency sweep from 100 to 0.1 rad/s. The mechanical properties of the 3DBP acellular structures 130 and cellular hydrogel scaffolds can be evaluated 1 day post printing and after swelling in PBS (pH 7.4) or in cell-culture media, respectively. Storage/loss moduli, complex viscosity, and elastic modulus can be measured at 1.99 Hz and reported to analyze the mechanical stability of the scaffolds.

In some examples, the microstructure characterization system 200 can perform a structural analysis procedure. For example, a tetrahedral meshing can be adopted to evaluate the mechanical properties of the 3DBP acellular structures 130 and cellular scaffolds 134 using ANSYS (e.g., Version: 2021 R2, Canonsburg, PA) which can be a step in the finite element analysis (FEA) process. This can involve dividing the geometry (stl. file) into small elements to approximate the continuous structure and performing the analysis of the complex geometry to obtain accurate and reliable results. Given the complex geometry of the structures and their resulting microstructures, an efficient way of simulating their mechanical parameters via loading can be to homogenize the material properties. For this purpose, Ansys Material Designer with the hybrid meshing feature, can be adopted to optimize microstructures and homogenize the material properties of complex these composite scaffolds containing cells and Au-NPs 210. To refine the mesh in certain regions of interest, such as in areas of high stress gradients, the number of nodes reported can be 144,485 and the number of elements can be 25,965. For running the simulations by applying a load of 9.3 N along the geometry's front face while fixing the back face, the following parameters can be adopted for 3DBP acellular structures 130 and cellular scaffolds 134, as depicted in Table 3. A Poisson's ratio of 0.5 can be assumed for hydrogels and polymers. The density, thermal conductivity, tensile yield and ultimate strength can be all assumed.

TABLE 3

| Parameters | Acellular | Cellular |
|---|---|---|
| Young's Modulus (Pa) | 44918.61 | 18487.02 |
| Bulk Modulus (Pa) | 7.4864e+05 | 3.0812e+05 |
| Shear Modulus (Pa) | 15073 | 6203.7 |

| | |
|---|---|
| Force (N) | 9.3 |
| Density (g/ml) | 1.053 |
| Thermal Conductivity (W/mK) | 0.06-0.30 |
| Tensile Yield Strength (kPa) | 70 |
| Tensile Ultimate Strength (kPa) | 40 |

The mechanical properties of the scaffolds can be examined using a static structural model in ANSYS. The resultant elastic stress, strain and total deformation can be evaluated and compared between 3DBP acellular structures 130 and cellular scaffolds 134, as well as with results obtained from rheological analysis and micro-CT scanning. All samples can be analyzed in triplicate, unless otherwise specified. Data can be represented as the mean+standard deviation. Comparison of the means of two independent samples can be performed by a t-test (e.g., GraphPad Prism 9) to determine if the averages of any two of the sample datasets compared showed significant difference in their values. P<0.05 can be considered statistically significant.

FIG. 3 depicts an example microstructure characterization system 300 including (A) live-dead florescent microscopy images 302 of AC16 cells treated with 1:10,000 Au-NPs and 1:100,000 Au-NPs respectively in 2D culture conditions. Live cells are represented as stained green whereas dead cells are represented as stained red by ethidium homodimer after 2 days in culture. The percentage of cell viability 304 is shown at (B) and can be calculated using the equations disclosed herein. In samples containing 1:10,000 Au-NPs and 1:100,000 Au-NPs the percentage of viability can be found to be equal to 74±6.4% and 93±10% respectively. From these results, the all Au-NP solutions 122 can be determined to be not cytotoxic.

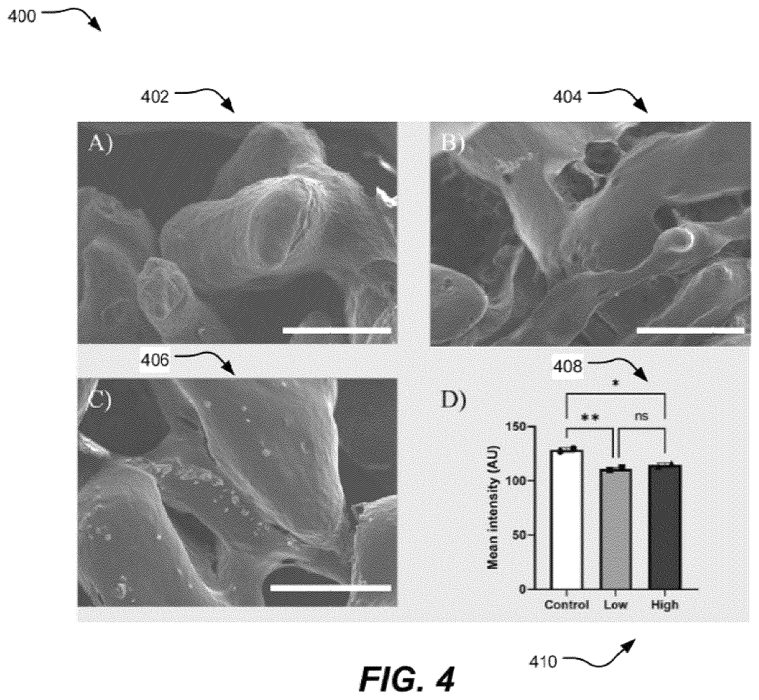

FIG. 4 depicts an example microstructure characterization system 400 including (A) the SEM micrographs 402 obtained for control samples (e.g., without Au-NPs) to observe the baseline resolution, contrast and optimum image quality of the scaffolds. (B) and (C) depict images obtained from samples containing low concentrations (1:100,000 dilution) 404 and high concentrations (1:100,000 dilution) 406 of Au-NPs respectively. All scale bars represent 1 mm in length. Portion (D) of FIG. 4 shows a quantitative comparison 408 of the mean intensity profile of the images obtained for samples containing varying amounts of Au-NPs 210. The results can reveal statistically significant differences in image contrast and intensity such that the microstructure characterization system 400 can differentiate between samples made without and with varying concentrations of Au-NP solutions. The samples containing high concentrations of Au-NPs can show visible nanoparticle aggregates on SEM micrographs with a reduced mean intensity of the sample, which can be analyzed using ImageJ. As shown in the (D) portion of FIG. 4 is a graph 410 with mean intensity profiles of all samples (n=2). *Represents p<0.05, ** represents p<0.01, and ns represents statistically not significant.

In some examples, the microstructure characterization system 400 can perform the micro-CT and/or the 3D structure visualization, as discussed herein. Samples containing 1:100,000 (low concentration) Au-NPs can generate a less clear image than when the micro-CT was performed on the scaffolds without any Au-NPs. Conversely, the microstructure characterization system 400 can obtain 595 micro-CT image slices from 3DBP acellular structures 130 containing 1:10,000 (high concentration) Au-NPs. Similarly, 657 sliced images can be obtained from scaffolds containing 1:10,000 (high concentration) Au-NPs with AC16 cells. Both sets of micro-CT images obtained can be processed using ImageJ Software to create a z-stacked projection.

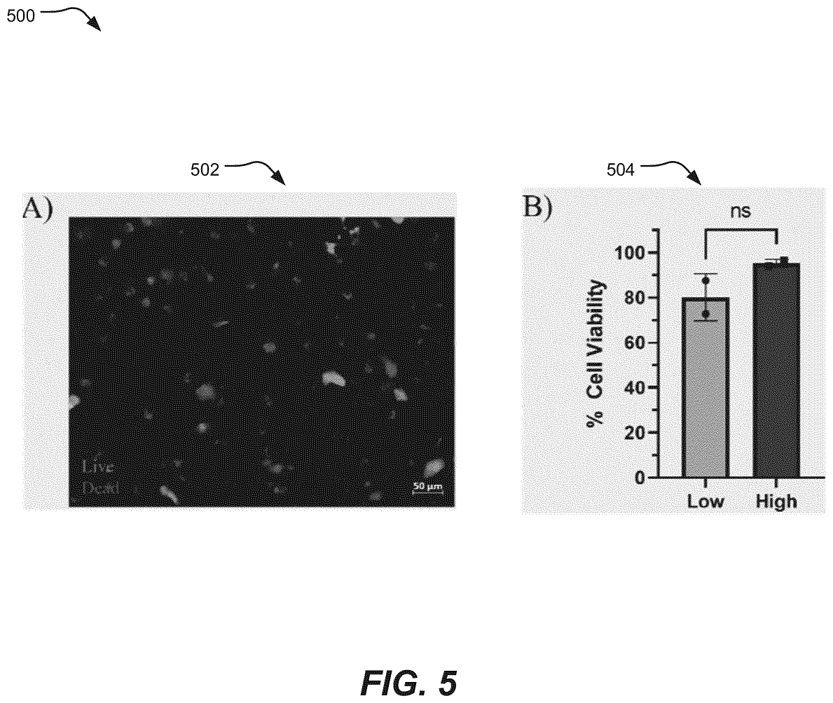

FIG. 5 depicts an example microstructure characterization system 500 including, at the (A) portion, the live-dead florescent microscopy images 502 of AC16 cells 3D bioprinted within gels containing high and low concentration of Au-NPs. Live cells are shown stained green by calcein AM and dead cells are shown stained red by ethidium homodimer. In samples containing high concentration of Au-NPs the percentage of cell viability can be found to be equal to 95±1.8% in comparison with the low concentration which depicted cell viability to be 80±10%. From these results, the microstructure characterization system 500 can determine that the Au-NPs are not cytotoxic to cells when they were in close contact in the 3D bioprinted gels. The (B) portion of FIG. 5 depicts a viability percentage 504 in 3D cell cultures (n=2).

FIG. 6 depicts an example microstructure characterization system 600 including stacked 3D projections 602 obtained for both samples (acellular and cellular), which can be processed to obtain the maximum intensity images. In some instances, the entire 3D scaffold structure can be observed in the (A) portion of FIG. 6 for the acellular sample, and the (B) portion for the scaffold containing AC16 cells, both samples containing 1:10,000 (high concentration) Au-NPs. These images can be processed using ImageJ to extract the visible scaffold structure. The (B) portion includes the portion of the sample with AC16 cardiomyocytes 604. Furthermore, the (C) portion shows the 3D STL. design 606 (20 mm×20 mm×1 mm) utilized for the 3D bioprinting process of scaffolds. The (D) portion shows an image outline 608 obtained for the acellular scaffold and the (E) portion depicts an image outline 610 of the scaffold containing AC16 cardiomyocytes. As can be observed in FIG. 6, addition of the Au-NPs can enhance the resolution and quality of the images, which can result in improved visualization of the microstructure of the 3D bioprinted scaffolds using micro-CT. As well, the micro-CT images as annotated with red dotted lines in the (D) and (E) portions obtained for both samples resembled the design utilized to fabricate these scaffold. The (D) portion shows micro-computed tomography results for acellular scaffold containing 1:10,000 (high concentration) Au-NPs, and the (E) portion scaffold containing 1:10,000 (high concentration) of Au-NPs and AC16 cardiomyocytes.

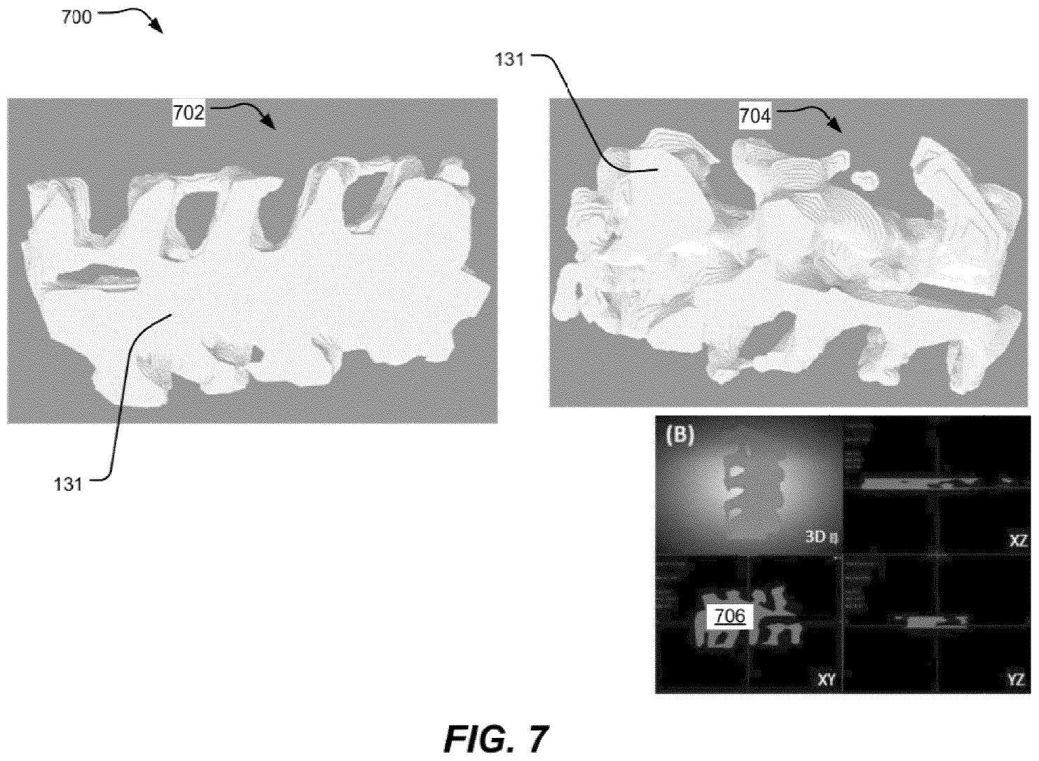

FIG. 7 depicts an example microstructure characterization system 700 including a virtual 3D reconstruction of both the 3DBP acellular structures 130 and cellular scaffolds 134. As can be observed from the (A) portion of FIG. 7, the acellular scaffold 702 appears to have more structural complexity as well as a more-intact structure. On the other hand, the (B) portion shows the cellular scaffold 704, which revealed structural dissolution owing to biodegradation due to the presence of the cells remodeling the scaffold, thereby affecting the quality and resolution of the 3D reconstruction for this sample. The results can show a higher definition of the overall structure of the 3DBP scaffolds due to inclusion of the Au-NPs.

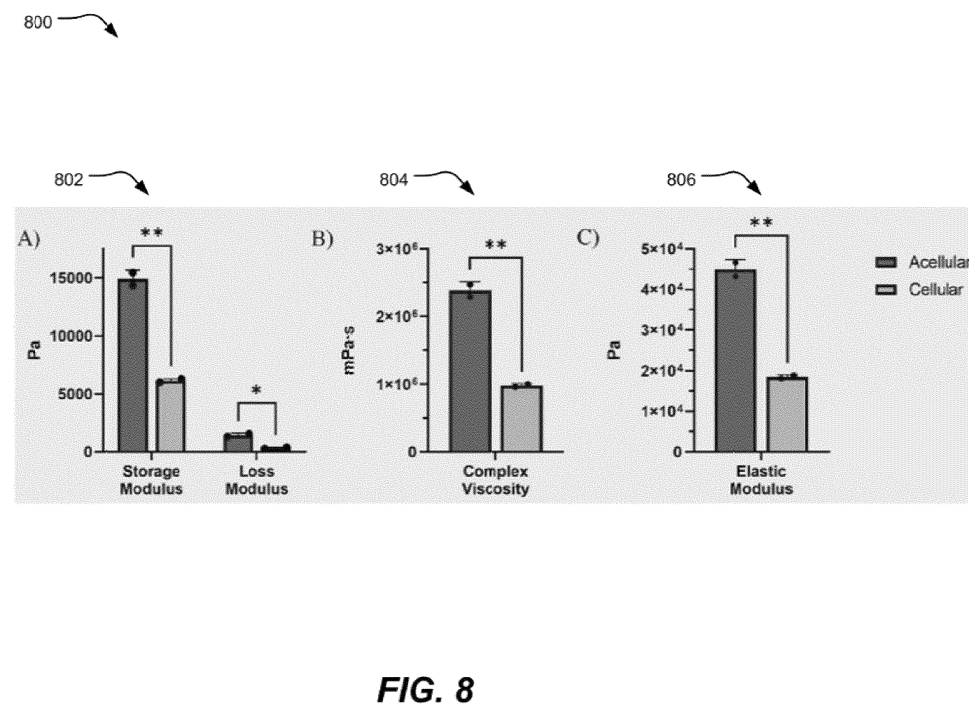

FIG. 8 depicts an example microstructure characterization system 800 which can evaluate the effect of encapsulating Au-NPs and cells on the mechanical properties of the hydrogels post-printing and 24-hours after in-vitro incubation. The microstructure characterization system 800 can perform an analysis which can be conducted on samples evaluated within the LVE range under a constant shear strain. (A) depicts a storage and loss modulus 802 for the 3DBP acellular structures 130 and cellular scaffolds. B) depicts the complex viscosity 804 for the acellular and cellular scaffolds 134. (C) depicts the calculated elastic modulus 806 for the 3DBP acellular structures 130 and cellular scaffolds 134 (n=2), where * represents p<0.05, and ** represents p<0.01.

In some examples, the results presented in the (A) portion depict a significantly higher storage/loss modulus in the 3DBP acellular structures 130 containing the Au-NPs 210 (14.9±0.8/1.43±0.19 KPa; p<0.05) as compared to those in cellular scaffolds 134 (6.15±0.19/0.32±0.08 KPa; p<0.05). Likewise, both the complex viscosity 804 (e.g., 2383100±129400.54/980820±28963.09 mPa·S; p<0.05) and elastic moduli 806 (e.g., 44.92±2.46/18.49±0.61 KPa; p<0.05) appeared to be greater in the 3DBP acellular structures 130 with Au-NPs 210 with respect to those in cellular scaffolds 134. The microstructure characterization system 800 can use these results to determine that the addition of Au-NPs enhances the overall mechanical properties of the 3DBP scaffolds. The inclusion of cells can be determined to lead to the biodegradation of the scaffolds during in vitro incubation as shown by the reduction in the values of storage/loss modulus in cellular scaffolds 134. Similarly, both the complex viscosity 804 and the elastic moduli 806 can be higher in magnitude in the 3DBP acellular structures 130 containing Au-NPs compared to those in the cellular scaffolds 134. These mechanical parameters can be further adopted for the mechanical analysis of the scaffolds. Comparison between the 3DBP acellular structures 130 and cellular scaffolds 134 can yield the following results as shown in Tables 4a, 4b, and 4c:

TABLE 4a
Total Deformation (m)

| | Minimum (m) | Maximum (m) | Average (m) |
|---|---|---|---|
| Acellular | 0 | 5.9394e−005 | 1.3292e−005 |
| Cellular | 0 | 1.4431e−004 | 3.2295e−005 |

TABLE 4b
Equivalent Elastic strain (m/m)

| | Minimum (m/m) | Maximum (m/m) | Average (m/m) |
|---|---|---|---|
| Acellular | 2.0667e−004 | 1.587e−002 | 5.4815e−003 |
| Cellular | 5.0215e−004 | 3.8561e−002 | 1.3319e−002 |

TABLE 4c
Equivalent stress (Pa)

| | Minimum (Pa) | Maximum (Pa) | Average (Pa) |
|---|---|---|---|
| Acellular | 8.79 | 712.88 | 234.22 |
| Cellular | 8.79 | 712.88 | 234.22 |

In some examples, the microstructure characterization system 800 can determine that the resultant elastic strain and total deformation can be greater in the cellular scaffolds 134 compared with the 3DBP acellular structures 130. These findings can corroborate with the rheological analysis data indicating the 3DBP acellular structures 130 can yield a higher elastic and storage moduli and therefore resist deformation and elongation. On the contrary, the cellular scaffolds 134 can retain more water because of the increased cellular content and depicted lower elastic and storage moduli. This can enable them to exhibit enhanced degrees of strain and deformation. Furthermore, the inclusion of cells can cause the scaffolds to be remodeled as revealed by the micro-CT results, which can also result in more strain and deformation of the structure to biodegrade and resulted in more strain and deformation.

In some examples, the role of scaffolds in tissue engineering as a substrate can mimic the native extracellular matrix (ECM) and can control and influence cell attachment, proliferation and differentiation. The response of a scaffold can be significantly influenced by the properties and composition of its constituents, or its microstructure. Additionally, for biomaterial scaffolds, the microstructure can pose as a mechanical cue, which can influence cell behaviors, and control key functions at the molecular and cellular levels. The microstructures disclosed herein can play a role in the control of the 3D bioprinted scaffold's mechanical properties, and variable microstructures can be produced by altering the gel parameters. The effect of the chemical crosslinking (gelling) parameters on the viscoelastic and diffusion properties of the scaffolds as well as other structural parameters (e.g., pore size, extent of porosity, average molecular weight of the polymer chain between neighboring crosslinks, crosslinking density) can all affect the resultant microstructure and overall mechanical properties. In some instances, a size, shape, or diameter value of the Au-NPs 210 can correspond to a type of cell attaching to the microstructure to optimize cell growth. For example, based on different cell sensitivities, each different cell type can have a different optimal gold nanoparticle size or concentration in terms of cell viability. The different cell types can also correspond to particular types of coatings suitable for the particular cells of the microstructure. This aspect of dynamic microstructure modulation can affect cell fate and turnover. Thus, the systems disclosed herein can perform a process of layer-by-layer 3D printing followed by chemical crosslinking at the microstructure scale in way that improves cell viability. Furthermore, additive manufacturing can generate anisotropic bulk material properties from an isotropic build material. Since precise and reliable prediction of macroscale material properties from the microscale can be provided by the systems disclosed herein, the microstructure can be optimized to obtain a resultant tissue structure that is functionally robust.

The systems disclosed herein can adopt the micro-CT technique for imaging of 3DBP scaffolds embedded with Au-NPs microstructure evaluation. Micro-CT can be a non-destructive imaging method that uses X-rays to rapidly digitize samples in three dimensions. The systems can use X-ray micro-CT scanners with sub-micron resolution. Limitation of micro-CT scanners in the low contrast of soft tissues due to low X-ray absorption can be overcome using contrast agents, as discussed herein. The inclusion of a contrast agent such as Au-NPs presents a non-destructive evaluation approach which has the potential to enhance the biomanufacturing workflow of these tissue engineered structures for clinical applications while avoiding the need to stain them, as stains can alter the characteristics of the tissues.

In some examples, benefits of using micro-CT imaging can include the generation of a series of projection images (e.g., radiographs) that can be reconstructed into cross-section images using reconstruction algorithms as detailed in the image acquisition workflow. 3D geometric morphometric methods via mechanical modeling and simulation can be applied in addition for comparisons between different results through the creation of surface models and the application of specified landmarks on them.

In some scenarios, from the contrast enhancement using gold nanoparticles perspective, colloidal stability and dispersion of the Au-NPs in the 3D bioprinted scaffolds can provide uniform contrast throughout the structure during micro-CT imaging. In some examples, minor aggregations of PEGylated gold nanoparticles can occur, although it may not significantly alter the micro-CT imaging quality. The dispersion of gold nanoparticles in the 3D bioprinting matrix can be considered random since the nanoparticles can be mixed into the matrix before bioprinting. The diffusion of gold nanoparticles can be limited due to the high viscosity of the 3D bioprinted material, and/or they may become concentrated locally by the 3D printing process or relocated in the biomaterial post-printing by the embedded cells. Some strategies to increase, better control, and/or maintain the uniform dispersion of gold nanoparticles throughout the 3D bioprinted material can include the addition of chemical moieties on the gold nanoparticle surface to anchor them covalently to the bio-matrix and/or further advancements in implementing the 3D bioprinting process for selective deposition and preferential alignment of nanoparticles.

The size of gold nanoparticle and concentration can also be used to control the amount of micro-CT contrast. Micro-CT contrast can increase proportionally to both nanoparticle size and concentration. There can be a trade-off between using larger size gold nanoparticles versus using higher concentrations of smaller size gold nanoparticles for more micro-CT imaging contrast. Larger size gold nanoparticles can have an increased likelihood to disrupt the native microstructure of the bioprinted scaffold due to modified mechanical properties of the nanoparticle-laden composite biomaterial and being similar sized to the micronozzle in the 3D printer. That said, in some scenarios, lower concentration can be used to achieve high and sufficient micro-CT imaging contrast. On the other hand, smaller size gold nanoparticles can be less likely to disrupt the native microstructure of the bioprinted scaffolds, but may use higher concentrations which, in turn may cause cellular toxicity or disrupt the cellular homeostasis in the co-deposited cells. As such, the systems disclosed herein can perform optimization of different concentrations of gold nanoparticles to compare the image resolution, quality, and the visibility of the microstructure of hydrogels.

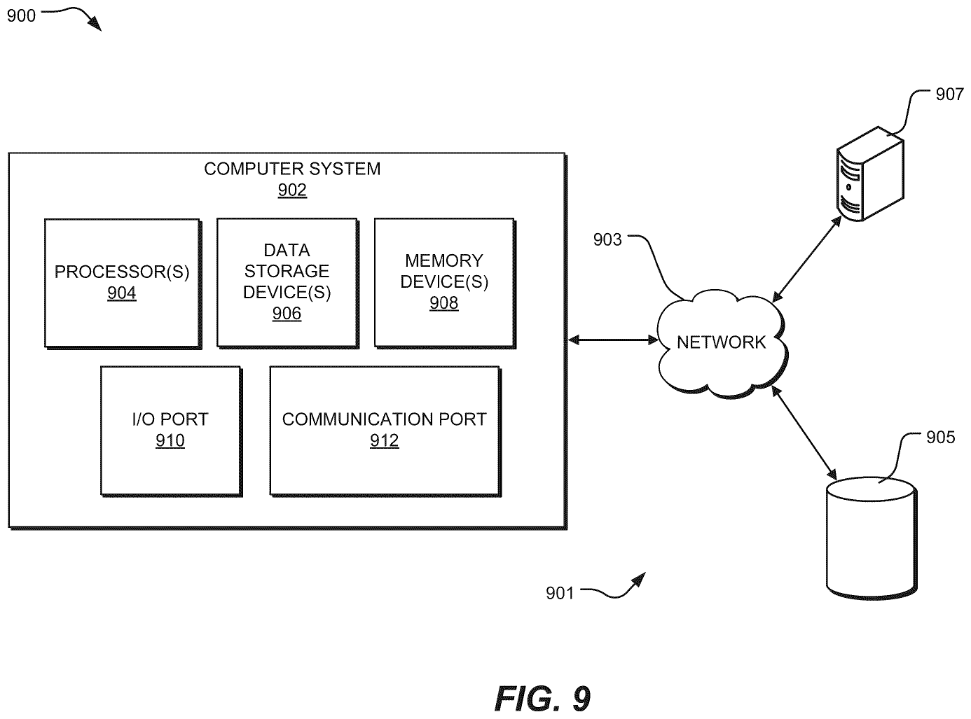

FIG. 9 depicts a microstructure characterization system 900 in an example network environment 901. As depicted in FIG. 9, a network 903 can be used by one or more computing or data storage devices for implementing the microstructure characterization system 900 The microstructure characterization system 900 may generate one or more user interfaces (e.g., the front-end user interface) for interaction with various aspects of the network 903, such as the visual presentations of the 3D characterizations of the microstructures.

In one implementation, computing devices 902, one or more databases 905, and/or other network components or computing devices described herein are communicatively connected to the network 903. Examples of the computing devices 902 can include a terminal, personal computer, a smartphone, a tablet, a mobile computer, a workstation, and/or the like. The computing devices 902 may include a mobile device and/or can otherwise be associated with a provider system. For example, the computing devices 902 may include one or more provider servers executing service-based applications associated with service providers of the microstructure characterization system 900.

As shown in FIG. 9, a server 907 can host the network environment 901. In one implementation, the server 907 also hosts a website or an application that users may visit to access the microstructure characterization system 900. The server 907 may be one single server, a plurality of servers with each such server being a physical server or a virtual machine, or a collection of both physical servers and virtual machines. In another implementation, a cloud hosts one or more components of the microstructure characterization system 900. The microstructure characterization system 900, the computing devices 902, the server 907, and other resources connected to the network 903 may access one or more additional servers for access to one or more websites, applications, web services interfaces, and/or the like.

The computing system 902 may be a computer system capable of executing a computer program product to execute a computer process. Data and program files of the microstructure characterization system 900 may be input to the computing system 902, which reads the files and executes the programs therein. Some of the elements of the computing system 902 are shown in FIG. 9 including one or more hardware processors 904, one or more data storage devices 906, one or more memory devices 908, and/or one or more ports 910-912. Additionally, other elements that will be recognized by those skilled in the art may be included in the computing system 902. Various elements of the computing system 902 may communicate with one another by way of one or more communication buses, point-to-point communication paths, or other communication means.

The processor 904 may include, for example, a central processing unit (CPU), a microprocessor, a microcontroller, a digital signal processor (DSP), a graphics processing unit (GPU), and/or one or more internal levels of cache. There may be one or more processors 904, such that the processor 904 includes a single central-processing unit, or a plurality of processing units capable of executing instructions and performing operations in parallel with each other, commonly referred to as a parallel processing environment.

The computing system 902 may be a conventional computer, a distributed computer, or any other type of computer, such as one or more external computers made available via a cloud computing architecture. The presently described technology is optionally implemented in software stored on the data storage device(s) 906, stored on the memory device(s) 908, and/or communicated via one or more of the ports 910-912, thereby transforming the computing system 902 in FIG. 9 to a special purpose machine for implementing the operations described herein, and providing many practical applications for the technology.

The one or more data storage devices 906 may include any non-volatile data storage device capable of storing data generated or employed within the computing system 902, such as computer-executable instructions for performing a computer process, which may include instructions of both application programs and an operating system (OS) that manages the various components of the computing system 902. The data storage devices 906 may include, without limitation, magnetic disk drives, optical disk drives, solid state drives (SSDs), flash drives, and the like. The data storage devices 906 may include removable data storage media, non-removable data storage media, and/or external storage devices made available via a wired or wireless network architecture with such computer program products, including one or more database management products, web server products, application server products, and/or other additional software components. Examples of removable data storage media include Compact Disc Read-Only Memory (CD-ROM), Digital Versatile Disc Read-Only Memory (DVD-ROM), magneto-optical disks, flash drives, and the like. Examples of non-removable data storage media include internal magnetic hard disks, SSDs, and the like. The one or more memory devices 908 may include volatile memory (e.g., dynamic random-access memory (DRAM), static random-access memory (SRAM), etc.) and/or non-volatile memory (e.g., read-only memory (ROM), flash memory, etc.).

Computer program products containing mechanisms to effectuate the systems and methods in accordance with the presently described technology may reside in the data storage devices 906 and/or the memory devices 908, which may be referred to as machine-readable media or computer-readable media. It will be appreciated that machine-readable media may include any tangible non-transitory medium that is capable of storing or encoding instructions (e.g., computer-readable instructions) to perform any one or more of the operations of the present disclosure for execution by a machine or that is capable of storing or encoding data structures and/or modules utilized by or associated with such instructions. Machine-readable media may include a single medium or multiple media (e.g., a centralized or distributed database, and/or associated caches and servers) that store the one or more executable instructions or data structures.

In some implementations, the computing system 902 includes one or more ports, such as the input/output (I/O) port 910 and the communication port 912, for communicating with other computing, network, or vehicle devices. It will be appreciated that the ports 910-912 may be combined or separate and that more or fewer ports may be included in the computing system 902.

The I/O port 910 may be connected to an I/O device, or other device, by which information is input to or output from the computing system 902. Such I/O devices may include, without limitation, one or more input devices, output devices, and/or environment transducer devices.

In one implementation, the input devices convert a human-generated signal, such as, human voice, physical movement, physical touch or pressure, and/or the like, into electrical signals as input data into the computing system 902 via the I/O port 910. The output devices may convert electrical signals received from computing system 902 via the I/O port 910 into signals that may be sensed as output by a human, such as sound, light, and/or touch. The input device may be an alphanumeric input device, including alphanumeric and other keys for communicating information and/or command selections to the processor 904 via the I/O port 910. The input device may be another type of user input device including, but not limited to: direction and selection control devices, such as a mouse, a trackball, cursor direction keys, a joystick, and/or a wheel; one or more sensors, such as a camera, a microphone, a positional sensor, an orientation sensor, a gravitational sensor, an inertial sensor, and/or an accelerometer; and/or a touch-sensitive display screen (“touchscreen”). The output devices may include, without limitation, a display, a touchscreen, a speaker, a tactile and/or haptic output device, and/or the like. In some implementations, the input device and the output device may be the same device, for example, in the case of a touchscreen.

The environment transducer devices convert one form of energy or signal into another for input into or output from the computing system 902 via the I/O port 910. For example, an electrical signal generated within the computing system 902 may be converted to another type of signal, and/or vice-versa. In one implementation, the environment transducer devices sense characteristics or aspects of an environment local to or remote from the computing device 902, such as, light, sound, temperature, pressure, magnetic field, electric field, chemical properties, physical movement, orientation, acceleration, gravity, and/or the like. Further, the environment transducer devices may generate signals to impose some effect on the environment either local to or remote from the example computing device 902, such as, physical movement of some object (e.g., a mechanical actuator), heating or cooling of a substance, adding a chemical substance, and/or the like.

In one implementation, a communication port 912 is connected to a network by way of which the computing system 902 may receive network data useful in executing the methods and systems set out herein as well as transmitting information and network configuration changes determined thereby. Stated differently, the communication port 912 connects the computing system 902 to one or more communication interface devices configured to transmit and/or receive information between the computing system 902 and other devices by way of one or more wired or wireless communication networks or connections. Examples of such networks or connections include, without limitation, Universal Serial Bus (USB), Ethernet, Wi-Fi, Bluetooth®, Near Field Communication (NFC), Long-Term Evolution (LTE), and so on. One or more such communication interface devices may be utilized via the communication port 912 to communicate one or more other machines, either directly over a point-to-point communication path, over a wide area network (WAN) (e.g., the Internet), over a local area network (LAN), over a cellular (e.g., third generation (3G) fourth generation (4G), or fifth generation (5G)) network, or over another communication means. Further, the communication port 912 may communicate with an antenna or other link for electromagnetic signal transmission and/or reception.

In an example implementation, microstructure characterization system software and other modules and services may be embodied by instructions stored on the data storage devices 906 and/or the memory devices 908 and executed by the processor 904.

In the present disclosure, the methods disclosed may be implemented as sets of instructions or software readable by a device. Further, it is understood that the specific order or hierarchy of steps in the methods disclosed are instances of example approaches. Based upon design preferences, it is understood that the specific order or hierarchy of steps in the method can be rearranged while remaining within the disclosed subject matter.

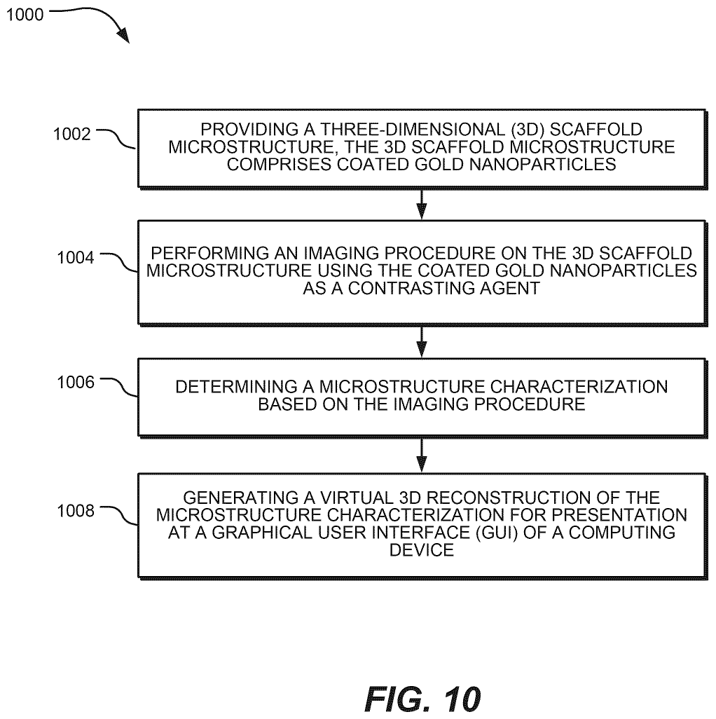

FIG. 10 depicts example operations of a method 1000 for microstructure characterization. The method 1000 shown in FIG. 10 can be performed by any of the systems 100-900 depicted in FIGS. 1-9. In some examples, at operation 1002, the method 1000 can provide a three-dimensional (3D) scaffold microstructure, the 3D scaffold microstructure comprising coated gold nanoparticles. At operation 1004, the method 1000 can perform an imaging procedure on the 3D scaffold microstructure using the coated gold nanoparticles as a contrasting agent. At operation 1006, the method 1000 can determine a microstructure characterization based on the imaging procedure. At operation 1008, the method can generate a virtual 3D reconstruction of the microstructure characterization for presentation at a graphical user interface (GUI) of a computing device.

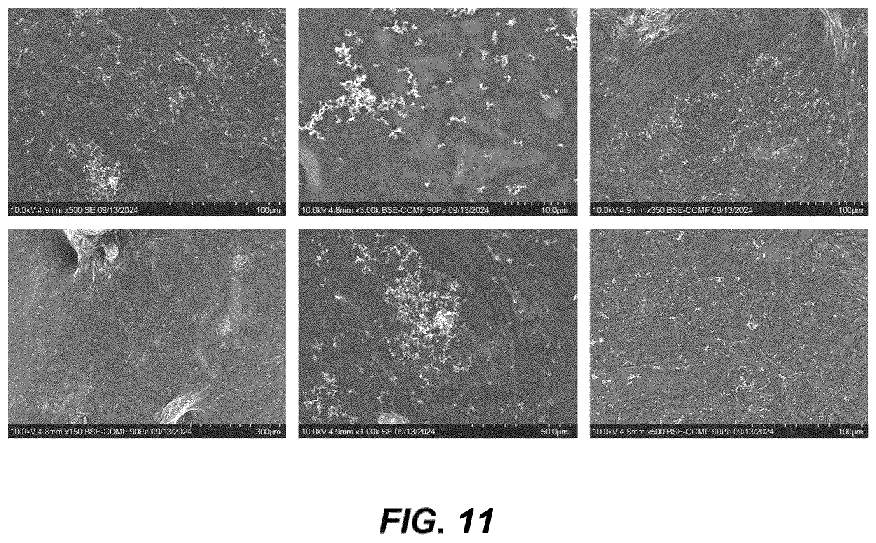

In one implementation, 3D bioprinted scaffolds composed of 5% (w/v) gelatin and 7% (w/v) medium viscosity alginic acid were mixed with 1 μL of stock solution of 200 nm core-sized gold nanoparticles (Au-NPs) per 1 mL of hydrogel solution. Scanning Electron Microscopy (SEM) images were obtained by utilizing a Hitachi SU3500 Variable-Pressure Scanning Electron Microscope (Santa Clara, CA, United States). Results are shown in FIG. 11 and demonstrate a higher contrast of the surface of the scaffolds.

Next, the biocompatibility of the Au-NPs was assessed. For this, AC16 human cardiomyocyte cell lines (SCC109, EMD Millipore, MA) were cultured in Dulbecco's Modified Eagle's Medium/Nutrient 137 Mixture (DMEM/F12, Sigma Cat. No. D6434, St. Louis, MO, United States), 10% FBS (EMD Millipore Cat. No. ES-009-B), and 1× penicillin-streptomycin solution (EMD Millipore Cat. No. TMS-AB2-C). One μL of the Au-NP solution studied (for 200 nm and 60 nm sized nanoparticle solutions) was added into a twelve-well culture plate containing AC16 cells, with a density of 60,000 cells per well, and cultured for 2 days in an incubator under standard cell culture conditions. Cytotoxicity of the Au-NPs was measured using a Live-Dead Assay Kit (Thermo Fisher Scientific, USA) according to the protocol provided by the vendor. Calcein AM (green) stained live cells, while ethidium homodimer (red) was used to stain dead cells after being incubated in the samples for 45 minutes at room temperature (RT) (25° C.). Live-dead assay images collected were analyzed by using ImageJ to determine cell viability. Live and dead cells were counted utilizing the cell-counter tool on ImageJ. % Cell viability (% live cells) versus % of dead cells was quantified using the following formula:

$$no . of live / dead cells \% = \frac{number of live or dead cells}{total numbers of live + dead cells} \times 100$$

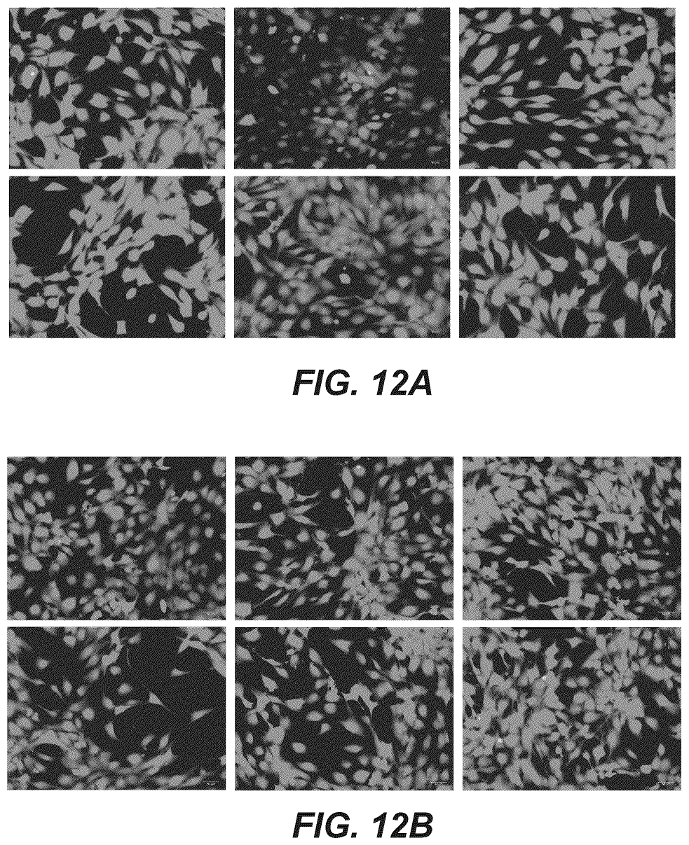

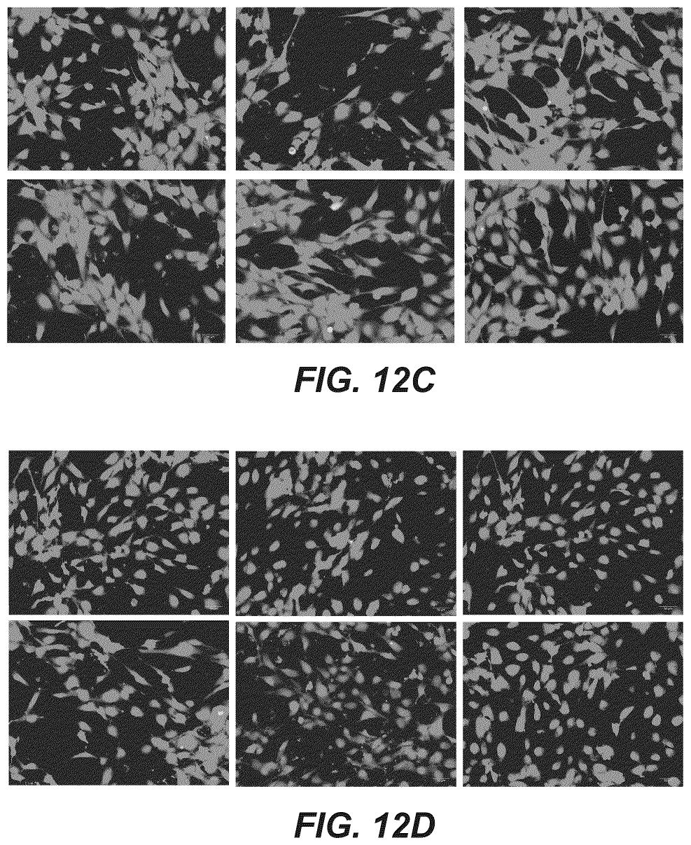

As seen in FIGS. 12A-12D and Tables 5 and 6, all samples showed a percentage of viability of at least 95%, which indicates that the gold nanoparticles are not cytotoxic. Cell viability results from the images are detailed in Table 5, and further summarized in the following Table 6:

TABLE 5
Results of each image analyzed.
Live Dead Assay

| Sample/Image Analyzed | Number of | Number of | Total | % Viability |
|---|---|---|---|---|
| Control | 88 | 1 | 89 | 98.88% |
| Control 2 | 65 | 0 | 65 | 100.00% |
| Control 3 | 69 | 1 | 70 | 98.57% |
| 200 nm well 1 | 116 | 6 | 122 | 95.08% |
| 200 nm well 1-B | 84 | 2 | 86 | 97.67% |
| 200 nm well 1-C | 67 | 1 | 68 | 98.53% |
| 200 nm well 2 | 79 | 0 | 79 | 100.00% |
| 200 nm well 2-B | 91 | 5 | 96 | 94.79% |
| 200 nm well 2-C | 113 | 11 | 124 | 91.13% |
| 200 nm 1-1000 Well 3 | 102 | 4 | 106 | 96.23% |
| 200 nm 1-1000 Well 3-C | 57 | 4 | 61 | 93.44% |
| 200 nm 1-1000 Well 3-B | 106 | 5 | 111 | 95.50% |
| 200 nm 1-1000 Well 2 | 130 | 4 | 134 | 97.01% |
| 200 nm 1-1000 Well 2-B | 124 | 2 | 126 | 98.41% |
| 200 nm 1-1000 Well 2-C | 129 | 7 | 136 | 94.85% |
| 200 nm 1-1000 Well 1 | 94 | 2 | 96 | 97.92% |
| 200 nm 1-1000 Well 1-C | 134 | 2 | 136 | 98.53% |
| 200 nm 1-1000 Well 1-B | 115 | 3 | 118 | 97.46% |
| 200 nm 1-10,000 Well 2 | 79 | 5 | 84 | 94.05% |
| 200 nm 1-10,000 Well 2-C | 110 | 5 | 115 | 95.65% |
| 200 nm 1-10,000 Well 2-B | 56 | 4 | 60 | 93.33% |
| 200 nm 1-10,000 Well 1 | 84 | 4 | 88 | 95.45% |
| 200 nm 1-10,000 Well 1-C | 106 | 1 | 107 | 99.07% |
| 200 nm 1-10,000 Well 1-B | 109 | 6 | 115 | 94.78% |
| 60 nm 1-5000 Well 1-B | 105 | 5 | 110 | 95.45% |
| 60 nm 1-5000 Well 1-C | 116 | 5 | 121 | 95.87% |
| 60 nm 1-5000 Well 1-D | 105 | 7 | 112 | 93.75% |
| 60 nm 1-5000 Well 1 | 71 | 3 | 74 | 95.95% |
| 60 nm 1-5000 Well 2-B | 84 | 1 | 85 | 98.82% |
| 60 nm 1-5000 Well 2-C | 119 | 4 | 123 | 96.75% |
| 60 nm 1-5000 Well 2 | 82 | 2 | 84 | 97.62% |
| 60 nm 1-5000 Well 3-B | 101 | 3 | 104 | 97.12% |
| 60 nm 1-5000 Well 3-C | 109 | 2 | 111 | 98.20% |
| 60 nm 1-5000 Well 3 | 75 | 4 | 79 | 94.94% |

TABLE 6
Summary of the cell viability results.

| | AVG | STDEV |
|---|---|---|
| Control | 99.15% | 0.752% |
| 200 nm | 96.20% | 3.193% |
| 200 nm 1-1000 | 96.59% | 1.733% |
| 200 nm 1-10,000 | 95.39% | 1.999% |
| 60 nm 1-5000 | 96.45% | 1.555% |

While specific implementations are discussed, it should be understood that this is done for illustration purposes only. A person skilled in the relevant art will recognize that other components and configurations may be used without parting from the spirit and scope of the presently disclosed technology. Any of the components of the microstructure characterization system(s) 100-900 or method 1000 can be combined with any other components of the microstructure characterization system(s) 100-900 or method 1000. The description and drawings are illustrative and are not to be construed as limiting. Numerous specific details are described to provide a thorough understanding of the presently disclosed technology. However, in certain instances, well-known or conventional details are not described in order to avoid obscuring the description. References to one or an implementation in the presently disclosed technology can be references to the same implementation or any implementation; and such references mean at least one of the implementations.

Reference to “one implementation” or “an implementation” means that a particular feature, structure, or characteristic described in connection with the implementation is included in at least one implementation of the presently disclosed technology. The appearances of the phrase “in one implementation” in various places in the specification are not necessarily all referring to the same implementation, nor are separate or alternative implementations mutually exclusive of other implementations. Moreover, various features are described which may be exhibited by some implementations and not by others.

The terms used in this specification generally have their ordinary meanings in the art, within the context of the presently disclosed technology, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only and is not intended to further limit the scope and meaning of the presently disclosed technology or of any example term. Likewise, the presently disclosed technology is not limited to various implementations given in this specification.

Without intent to limit the scope of the presently disclosed technology, examples of instruments, apparatus, methods and their related results according to the implementations of the presently disclosed technology are given below. Unless otherwise defined, technical and scientific terms used herein have the meaning as commonly understood by one of ordinary skill in the art to which the presently disclosed technology pertains.

What is claimed is:

1. A method to characterize a microstructure, the method comprising:
providing a three-dimensional (3D) scaffold microstructure, the 3D scaffold microstructure comprising coated gold nanoparticles;
performing an imaging procedure on the 3D scaffold microstructure using the coated gold nanoparticles as a contrasting agent;
determining a microstructure characterization based on the imaging procedure; and
generating a virtual 3D reconstruction of the microstructure characterization for presentation at a graphical user interface (GUI) of a computing device.

2. The method of claim 1,
wherein,
the providing of the 3D scaffold microstructure includes bioprinting the 3D scaffold microstructure using a 3D bioprinter.

3. The method of claim 1,
wherein,
the 3D scaffold microstructure includes an alginate-gelatin hydrogel.

4. The method of claim 1,
wherein,
the coated gold nanoparticles include methoxy-poly(ethylene glycol) (methoxy-PEG) coated gold nanoparticles.

5. The method of claim 1, further comprising:
determining, based on the microstructure characterization, a degree of pore interconnectivity affecting cell distribution, attachment, or growth in the 3D scaffold microstructure.

6. The method of claim 1,
wherein,
the imaging procedure includes a micro-computed tomography (micro-CT) procedure.

7. The method of claim 1, further comprising:
determining a size of gold nanoparticles by performing at least one of an ultraviolet (UV)-visible spectroscopy procedure, a dynamic light scattering (DLS) procedure, a zeta potential measurement procedure, a scanning electron microscopy procedure (SEM), or a transmission electron microscopy (TEM) procedure.

8. The method of claim 1,
wherein,
the providing of the 3D scaffold microstructure includes:
obtaining a solution of 2 kDa methoxy-poly(ethylene glycol) (methoxy-PEG) coated gold nanoparticles,
mixing the solution of the 2 kDa methoxy-PEG coated gold nanoparticles with a saline solution, a first concentration of gelatin, and a second concentration of sodium alginate to form a resultant solution, and
providing the resultant solution to a 3D bioprinter operating under one or more predefined 3D bioprinting parameters.

9. The method of claim 8,
wherein,
the first concentration of gelatin is between 4-6% (w/v) gelatin and the second concentration of sodium alginate is between 6-8% (w/v) sodium alginate.

10. The method of claim 8,
wherein,
the one or more predefined 3D bioprinting parameters includes at least one of a nozzle size of 22G, a printing speed of about 1 mm/s, a pressure of about 25 kPA, or a temperature of about 25° C.

11. The method of claim 1, further comprising:
optimizing a concentration of the coated gold nanoparticles in the 3D scaffold microstructure to optimize cell growth while optimizing imaging contrast.

12. A method to characterize a microstructure, the method comprising:
providing a three-dimensional (3D) scaffold microstructure, the 3D scaffold microstructure comprising methoxy-poly(ethylene glycol) (methoxy-PEG) coated gold nanoparticles;
performing an imaging procedure on the 3D scaffold microstructure using the methoxy-PEG coated gold nanoparticles as a contrasting agent;
determining a microstructure characterization based on the imaging procedure; and
generating a virtual 3D reconstruction for presentation at a graphical user interface (GUI) of a computing device is based on the microstructure characterization.

13. The method of claim 12,
wherein,
the methoxy-PEG coated gold nanoparticles have a core diameter of between 15 nm and 200 nm.

14. The method of claim 12,
wherein,
the methoxy-PEG coated gold nanoparticles have a core diameter of about 60 nm.

15. The method of claim 12,
wherein,
the providing of the 3D scaffold microstructure includes:
preparing a solution of 2 kDa methoxy-PEG coated gold nanoparticles,
diluting the solution to form varying concentrations of the solution, and
using the varying concentrations of the solution to form, via a 3D bioprinter, a plurality alginate-gelatin hydrogels having varying concentrations of the 2 kDa methoxy-PEG coated gold nanoparticles.

16. A method to characterize a microstructure, the method comprising:
providing a three-dimensional (3D) scaffold microstructure, the 3D scaffold microstructure comprising methoxy-poly (ethylene glycol) (methoxy-PEG) coated gold nanoparticles;
performing an imaging procedure on the 3D scaffold microstructure, using the methoxy-PEG coated gold nanoparticles as a contrasting agent, to determine a microstructure characterization of the 3D scaffold microstructure; and
causing a virtual 3D reconstruction to be presented at a graphical user interface (GUI) of a computing device based on the microstructure characterization.

17. The method of claim 16,
wherein,
the providing of the 3D scaffold microstructure includes bioprinting, using a 3D bioprinter, the 3D scaffold microstructure from a solution of the methoxy-PEG coated gold nanoparticles, gelatin, and sodium alginate.

18. The method of claim 16,
wherein,
the method includes a gold nanoparticle characterization procedure including at least one of an ultraviolet (UV)-visible spectroscopy procedure, a dynamic light scattering (DLS) procedure, a zeta potential measurement procedure, or a transmission electron microscopy (TEM) procedure.

19. The method of claim 16, further comprising:
determining, based on the microstructure characterization, a degree of pore interconnectivity affecting cell distribution, attachment, or growth in the 3D scaffold microstructure.

20. The method of claim 16, further comprising:
optimizing a concentration of the methoxy-PEG coated gold nanoparticles in the 3D scaffold microstructure to optimize cell growth while optimizing imaging contrast to yield an optimized concentration of the methoxy-PEG coated gold nanoparticles in a solution between a 0.022 optical density and a 2.2 optical density.
